(19) United States
(12) Patent Application Publication
(10) Pub. No.: US 2024/0197267 A1
(43) Pub. Date: Jun. 20, 2024

(54) CARDIAC GATED DIGITAL TOMOSYNTHESIS

(71) Applicant: CARESTREAM HEALTH, INC., Rochester, NY (US)

(72) Inventors: David H. FOOS, Webster, NY (US); Levon O. VOGELSANG, Webster, NY (US); Xiaohui WANG, Pittsford, NY (US)

(21) Appl. No.: 18/556,731

(22) PCT Filed: May 25, 2022
(86) PCT No.: PCT/US22/30804

§ 371 Date: Oct. 23, 2023

Related U.S. Application Data
(60) Provisional application No. 63/194,448, filed on May 28, 2021.

(51) Int. Cl. A61B 6/02; A61B 6/00; A61B 6/40; G06T 11/00

(52) U.S. Cl. CPC A61B 6/025; A61B 6/4007; A61B 6/486; G06T 11/005

(57) ABSTRACT

A plurality of radiographic cardiac images of a living patient are captured and grouped according to a cardiac phase depicted in each of the captured images. A 3D cardiac image is then reconstructed for each phase group. A 4D motion reconstruction is generated and displayed using the plurality of 3D reconstructions. A 3D reconstruction using all of the plurality of cardiac images may be used as an alignment check.

### BACKGROUND OF THE INVENTION

The subject matter disclosed herein relates to digital tomosynthesis imaging that enables the viewing of dynamically captured x-ray images of patient anatomy.

### BRIEF DESCRIPTION OF THE INVENTION

A plurality of radiographic cardiac images of a living patient are captured and grouped according to a cardiac phase depicted in each of the captured images. A 3D cardiac image is then reconstructed for each phase group. A 4D motion reconstruction is generated and displayed using the plurality of 3D reconstructions. A 3D reconstruction using all of the plurality of captured cardiac images may be used as an alignment constraint or alignment guide.

In one embodiment, a method is disclosed including capturing a plurality of radiographic cardiac images of a living patient. Each of the captured cardiac images is assigned to one of a plurality of phase groups according to the cardiac phase of each of the captured cardiac images. A 3D cardiac image is reconstructed for each of the phase groups. A 4D motion image may also be reconstructed using the reconstructed 3D cardiac images.

In another embodiment, a plurality of radiographic cardiac images of a living patient are captured over a plurality of cardiac cycles. The captured cardiac images are separated into one of a plurality of phase groups according to a cardiac phase of the captured cardiac images. A 3D cardiac image is reconstructed for each of the plurality of phase groups.

In another embodiment, a plurality of radiographic cardiac images of a living patient are captured at a rate of at least four radiographic images per cardiac cycle over at least four cardiac cycles. Each of the captured cardiac images are separated into one of at least four phase groups according to a cardiac phase of the captured cardiac images. A 3D cardiac image is reconstructed for each of the at least four phase groups.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a flow diagram of an exemplary method for reconstructing phased 3D and 4D tomosynthesis radiographic images;

FIGS. 2A-2C show an example time averaged reconstruction of a portion of a human heart, a phased reconstruction at full contraction, and a phased reconstruction at full expansion, respectively;

FIG. 3A-3B shows side by side human chest images of a reconstructed 4D digital tomosynthesis slice and a reconstructed 3D digital tomosynthesis slice; and

FIGS. 4A-4B are perspective views of two mobile digital radiography apparatuses.

### DETAILED DESCRIPTION OF THE INVENTION

This application claims priority to U.S. Patent Application Ser. No. 63/194,448, filed May 28, 2021, in the name of Foos et al., and entitled CARDIAC GATED DIGITAL TOMOSYNTHESIS, which is hereby incorporated by reference herein in its entirety.

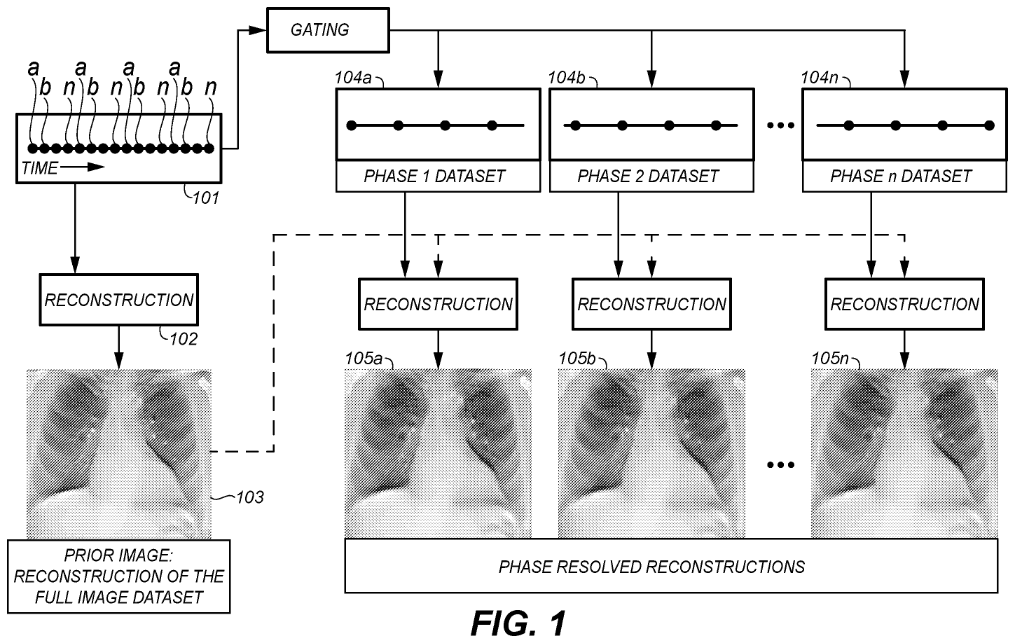

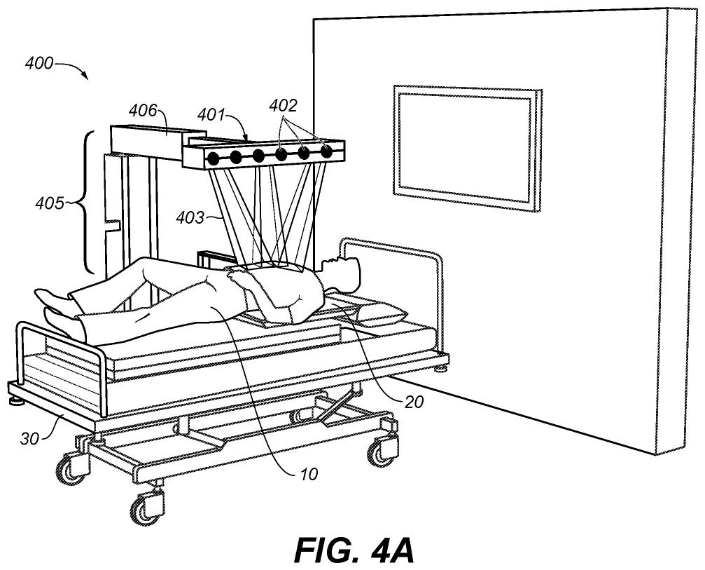

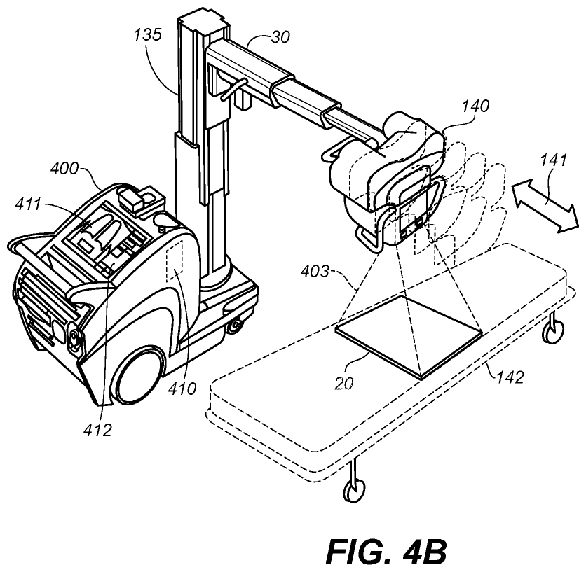

As illustrated in FIG. 1, the x-ray imaging method as disclosed herein includes acquiring a full set of digital x-ray tomosynthesis (DT) cardiac images 101 of a living patient, captured along a time line moving left-to-right at equal intervals. In the example representative full set of DT cardiac images 101 as illustrated in FIG. 1, sixteen cardiac images are captured which cover four complete cardiac cycles of expansion and contraction, resulting in four cardiac images per cardiac cycle. Of course, based on adjustment of programmed x-ray timing and desired imaging numbers and frequency, any number of images may be selected to be captured on an images-per-cycle basis and any number of desired heartbeat cycles may be covered. The images 101 may be acquired using a standard stationary tomosynthesis system which may include a source-and-detector pair revolving around a living patient's body, or the images 101 may be captured using a mobile tomosynthesis system or apparatus having a tube head that is movable over a range of imaging angles with respect to the stationary patient (FIG. 4B). In one embodiment, the mobile tomosynthesis system may include a stationary tube head having a plurality of cold cathode or carbon nanotube x-ray sources positioned over a range of imaging angles, with respect to the stationary patient, that are fired in a programmed sequence (FIG. 4A). A wired or wireless digital radiographic detector 20 may be positioned behind the patient to capture and transmit the set of tomosynthesis images 101.

The acquired full dataset of tomosynthesis images 101 are gated, or separated, and electronically stored into groups 104a, 104b . . . 104n according to a cardiac phase captured in each cardiac image. As an exemplary representation, images in group a may each be captured at a corresponding time early in a cardiac cycle, while images in group b are each captured at a corresponding later time in the same cardiac cycle as the immediately previous image a, and images in group n are each captured last in a corresponding cardiac cycle. As mentioned herein, any number of images may be programmed to be captured in each cardiac cycle. Thus, cardiac image groups 104a . . . 104n each correspond to a different cardiac phase and the cardiac images within each group all correspond to the same cardiac phase. According to one embodiment of the present invention, the full set of cardiac images 101 is reconstructed 102 to form a 3D cardiac image 103. As described herein, a 3D reconstruction may be generated using known tomosynthesis reconstruction techniques such as an iterative reconstruction algorithm. This results in a sufficiently sampled, temporally averaged 3D reconstruction 103. The separate cardiac phase groups 104a . . . 104n are each reconstructed to form a corresponding 3D image 105a . . . 105n, one 3D image per phase group representing a particular cardiac phase common to all cardiac images in the group. The full sufficiently sampled 3D reconstruction 103 may be used as a prior image alignment constraint.

The phase-specific 3D reconstructions 105a . . . 105n may then be individually displayed, according to any desired phase of the cardiac movement. One advantage of generating the phase specific reconstructions, as described herein, is reduced image blurring caused by movement of the heart during a cardiac cycle. A temporally averaged cardiac image combines images of the heart captured at various times during its rhythmic cyclic motion which introduces image blurring. The phase-specific 3D reconstructions 105a . . . 105n may also be displayed in a timed sequence to present the resulting 3D reconstructions in motion, otherwise known as displaying in 4D.

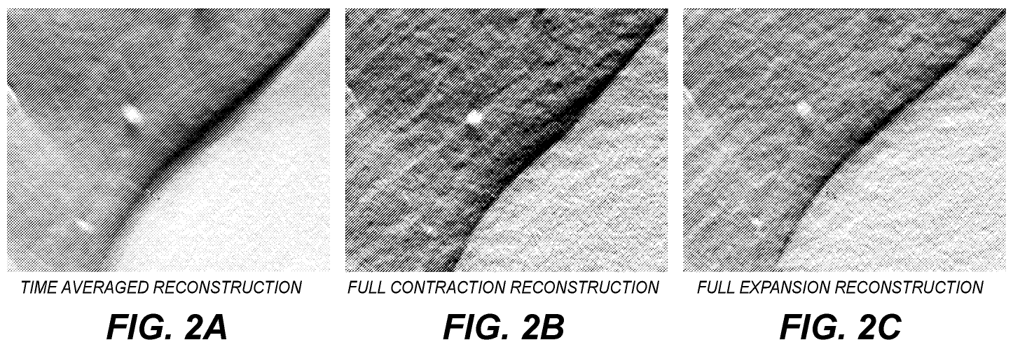

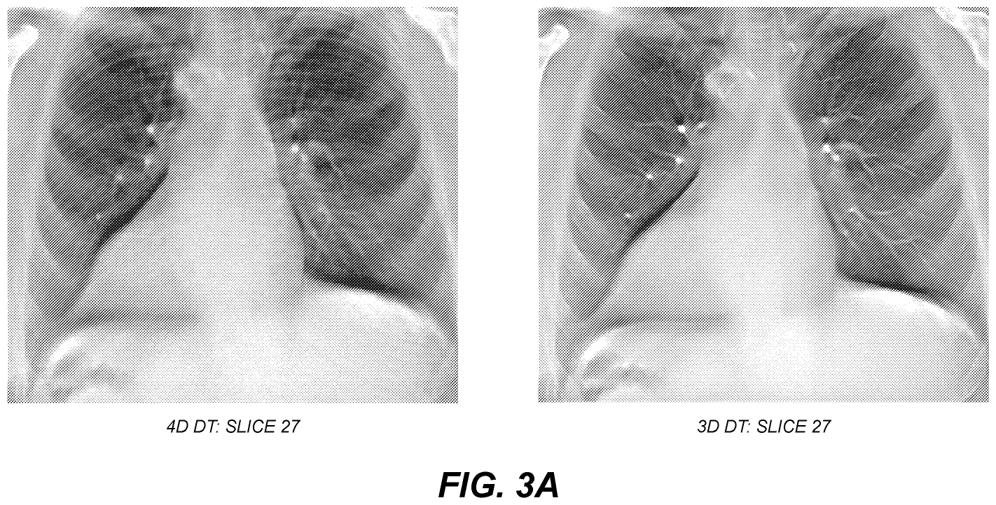

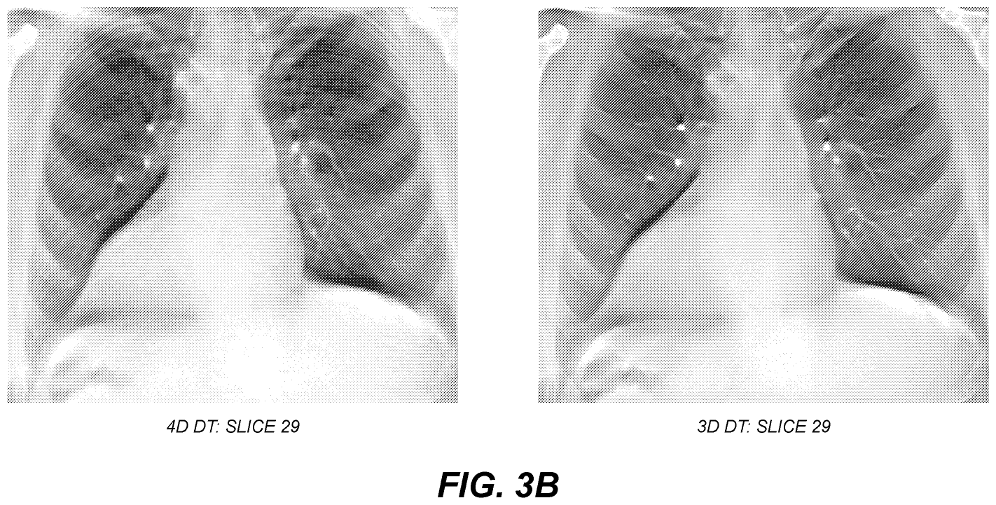

FIG. 2A presents a time averaged reconstruction of a portion of a cardiac tomosynthesis image. FIG. 2B presents a single phase reconstruction of the same portion of the cardiac tomosynthesis image at a full contraction phase. FIG. 2C a presents a single phase reconstruction of the same portion of the cardiac tomosynthesis image at a full expansion phase. FIGS. 3A-3B display image slices obtained from a 4D reconstruction (on the left in FIGS. 3A-3B) and image slices obtained from a 3D reconstruction (on the right in FIGS. 3A-3B).

FIG. 4A is a perspective view of a mobile radiography apparatus 400 guided into position at the bedside of a patient 10 in a medical imaging facility. The mobile radiography apparatus 400 may include a support member 405 having a horizontal section 406 that includes a tube head 401 attached thereto. The tube head 401 includes a plurality of activatable x-ray sources 402. In the embodiment shown in FIG. 4A, the horizontal section 406 may comprise a telescoping horizontal section 406 that extends outward at a fixed or variable distance from the support member 405. The horizontal section 406 may be configured to ride vertically up and down the support member 405 to a desired height for obtaining radiographic images of a patient 10. In another embodiment, the tube head 401 can be rotatably coupled to the horizontal section 406.

The perspective view of FIG. 4A shows mobile radiography apparatus 400 in position for digital tomosynthesis imaging of patient 10. Although one embodiment described herein illustrates use of a mobile radiography apparatus 400, a tube head 401, useable for tomosynthesis imaging, may be attached to a ceiling mounted stationary radiography apparatus such as an overhead tube head crane for in-room radiography imaging. The perspective view of FIG. 4A shows the tube head 401 having its linear array of x-ray sources 402 positioned somewhat along, i.e., parallel with, the length of the body of the patient 10. It may be preferred to position the tube head 401 such that the linear array of x-ray sources 402 are positioned cross-wise to the length of the body of the patient 10, such that a sequential firing of the array of x-ray sources 402 proceeds left-to-right, or vice versa, from the perspective of the patient 10. The patient 10 may be lying flat on a bed 30 with a DR detector 20 fitted behind the patient 10, with respect to the x-ray sources 402 in the tube head 401. There is no inherent mechanical linkage or coupling between x-ray tube head 401 and the detector 20. The x-ray sources 402 in the tube head 401 may be programmed to fire individually in any sequence desired. Corresponding tomosynthesis projection images of the patient 10 may be captured by the portable DR detector 20 and transmitted wirelessly to the mobile radiography apparatus 400 or to another processing system of the medical imaging facility.

The plurality of x-ray sources 402 within the tube head 401 are configured to emit a preselected plurality of individual x-ray beams 403 toward patient 10 and DR detector 20 to capture tomosynthesis projection images of a portion of the patient 10, as described herein. The DR detector 20 may be programmed to capture sequential images of patient 10 in coordination with a preselected and programmed sequential firing of x-ray sources 402 in the tube head 401. Because the x-ray sources 402 are arranged in a linear array, the radiographic images captured in detector 20 each correspond to an individual firing of one of the x-ray sources 402 of the array and each radiographic image so captured embodies a different imaging angle, which is useful for tomosynthesis purposes. All, or a subset, of the x-ray sources 402 may be programmed for activation in any sequence to emit an x-ray beam 403 to capture tomosynthesis images of a patient 10 using DR detector 20. The x-ray sources 402 may include cold cathode or carbon nanotube x-ray sources. Tube head 401 is shown having six x-ray sources 402, however, any other desired number of x-ray sources 402 may be included. The x-ray sources 402 may each be enclosed in a different vacuum chamber, or vacuum tube, or the x-ray sources 402 may all be enclosed by, or sealed in, a single vacuum chamber. The x-ray sources 402 are each configured to emit, when programmably activated, an x-ray beam 403 to generate projection x-ray images captured in DR detector 20. In the exemplary embodiment of FIG. 4A, the x-ray sources 402 may be fired one at a time in a programmed sequence, thereby emitting x-ray beams 32, one at a time, while the DR detector 20 captures one projection x-ray image for each firing of an x-ray source. In the exemplary embodiment of FIG. 4A, the x-ray sources 402 are shown to be arranged linearly within tube head 401, however, a suitable tube head housing may be used to enclose any shape or arrangement formed by x-ray sources 402. In actual operation, twelve x-ray sources 402 may be positioned in tube head 401 and configured in a similar manner as illustrated in FIG. 4A.

The perspective view of FIG. 4B shows an example mobile radiography apparatus 400 having a different type of support member 135, horizontal section 30, and tube head 140, attached thereto as compared to the mobile radiography apparatus 400 shown in FIG. 4A. Telescoping horizontal section 30 is extendible and retractable in a linear direction 141 away from and toward the support member 135. A patient may be positioned lying flat on the bed 142 over the detector 20. The DR detector 20 is fitted behind the patient with respect to the x-ray source in the tube head 140. There is no inherent mechanical linkage or alignment between x-ray tube head 140 and the detector 20. The x-ray source in the tube head 140 is moved to several imaging positions, or angles, relative to the portable DR detector 20 by extending and/or retracting the horizontal section 30. Tomosynthesis projection images of a patient are captured in the portable DR detector 20 at each imaging position of the tube head 140 as it is moved along the linear path 141. As shown in FIG. 4B, for linear travel of the tube head 140 it may be advantageous to use a telescoping motorized transport apparatus in the horizontal section 30. Thus, capturing successive tomosynthesis projection x-ray images of a patient at different relative angles may be achieved, as shown by the dashed line ghost images of tube head 140, using the x-ray source in tube head 140 and the detector 20.

A processing system 410 in the base of mobile radiography apparatus 400 provides a user interface on display 411 to allow a user to input operational control settings for x-ray imaging. The display 411 may also be used to present x-ray images captured by digital detector 20 via wireless digital communication therewith. The processing system 410 communicates digitally with tube head 140, 401, to control x-ray source timing, x-ray source selection, x-ray source firing and/or movement of the tube head 140, 410. A control panel 412 allows a user to select functions such as storing, transmitting, modifying, and printing of the captured images. For mobility, mobile radiography apparatus 400 has wheels and a handle grip to guide, or roll, the mobile radiography apparatus 400 to its intended location. A self-contained battery pack typically provides source power, eliminating the need for operation near a power outlet.

In one embodiment, the firing sequence of the x-ray tube head may be optimized to capture a cardiac phase of interest. The x-ray tube head 401 may be programmably timed to fire every 100 ms, using the multiple x-ray sources 402 one at a time in a timed sequence that is repeated over a time window of 6-9 seconds, for example. This will result in covering approximately 5-15 heartbeat cycles, depending on heart rate, in the full dataset by capturing 60-90 x-ray images. This full data set may then be used to reconstruct a 3D cardiac image 103 and separated into phase groups 104a-104n, as described herein, to reconstruct a number of phase specific 3D cardiac images. It may be useful to measure a patient's heart rate in order to determine a duration of one cardiac cycle in the patient. The cardiac cycle duration can then be divided by the number of images desired to be captured per cardiac cycle to determine the x-ray source timing and firing rate.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. Method comprising:
positioning an x-ray tube head and a digital radiographic detector about a patient to be radiographically imaged, the tube head comprising a plurality of x-ray sources;
firing the plurality of x-ray sources in a preselected timed sequence to capture a plurality of radiographic cardiac images of the patient over a plurality of cardiac cycles;
separating the captured cardiac images each into one of a plurality of phase groups according to a cardiac phase of said captured radiographic cardiac images; and
reconstructing a 3D cardiac image for each of the plurality of phase groups using the corresponding separated cardiac images.

2. The method of claim 1, further comprising displaying a 4D reconstruction using the reconstructed 3D cardiac images.

3. The method of claim 1, wherein the members of each phase group comprise an image of a common cardiac phase.

4. The method of claim 1, wherein the tube head comprises a linear array of x-ray sources.

5. The method of claim 1, wherein the step of firing the plurality of x-ray sources in a preselected sequence is repeated over a plurality of cardiac cycles.

6. The method of claim 1, wherein the step of firing the plurality of x-ray sources in a preselected sequence comprises capturing a plurality of radiographic cardiac images of the patient each at a different imaging angle.

7. Method comprising:
capturing a plurality of radiographic cardiac images of a living patient;
assigning each of the captured cardiac images into one of a plurality of phase groups according to a cardiac phase of said each of the captured cardiac images; and
reconstructing a 3D cardiac image for each of the plurality of phase groups using the corresponding assigned captured cardiac images.

8. The method of claim 7, further comprising displaying a 4D reconstruction using the reconstructed 3D cardiac images.

9. The method of claim 7, wherein each member of a phase group comprises a cardiac image of the living patient captured during the same cardiac phase.

10. Method comprising:
capturing a plurality of radiographic cardiac images of a living patient at a rate of at least four radiographic images per cardiac cycle over at least four cardiac cycles;
assigning each of the captured cardiac images into one of at least four phase groups according to a cardiac phase of said each of the captured cardiac images; and
reconstructing a 3D cardiac image for each of the plurality of phase groups using the corresponding captured cardiac images.

11. The method of claim 10, further comprising displaying a 4D reconstruction using the reconstructed 3D cardiac images.

12. The method of claim 10, wherein each member of a phase group comprises a cardiac image of the living patient captured during the same cardiac phase.
